(19) United States
(12) Patent Application Publication
(10) Pub. No.: US 2023/0226233 A1
(43) Pub. Date: Jul. 20, 2023

(54) ELECTRONIC DEVICES WITH ULTRAVIOLET LIGHT SOURCES

(71) Applicant: Hewlett-Packard Development Company, L.P., Spring, TX (US)

(72) Inventors: Hsiang-Ta KE, Taipei (TW); Yu-Hsing Kao, Taipei (TW); Hao-Hsuan Tsai, Taipei (TW)

(21) Appl. No.: 17/575,737

(22) Filed: Jan. 14, 2022

(51) Int. Cl. A61L 2/08; A61L 2/26; A61L 2/10

(52) U.S. Cl. CPC A61L 2/088; A61L 2/26; A61L 2/10; A61L 2202/11; A61L 2202/14

(57) ABSTRACT

In an example, an electronic device includes a housing and an input device exposed in an upper surface of the housing. The input device may include a transparent input interface. Further, the electronic device may include a transparent photocatalyst layer disposed on the transparent input interface, an ultraviolet (UV) light source disposed in the input device to emit light, and a conductive oxide layer disposed in the input device between the transparent input interface and the UV light source. Furthermore, the electronic device may include a controller to control a degree of transparency of the conductive oxide layer to permit the light to pass through the conductive oxide layer to contact the transparent photocatalyst layer.

### BACKGROUND

Electronic devices, such as laptop computers, convertible devices, or the like, may include a user interface that permits a user to interact with the electronic devices. An example user interface may include a keyboard, a touchscreen display panel, a touch pad, or the like. The user interface may enable users to input data into the electronic devices, for instance, either by touching a touch interface or by pressing keys. Such touch-based electronic devices may be shared by multiple users, for instance, in a public or private environment such as an enterprise, a hospital, a home, an educational establishment, or the like.

### BRIEF DESCRIPTION OF THE DRAWINGS

Examples are described in the following detailed description and in reference to the drawings, in which:

FIG. 1A is a cross-sectional side view of an example electronic device, including a controller to control a degree of transparency of a conductive oxide layer to permit light to contact a transparent photocatalyst layer;

FIG. 1B is a cross-sectional side view of the example electronic device of FIG. 1A, depicting additional features;

FIG. 2A is a cross-sectional side view of an example electronic device, including a controller to control a degree of transparency of a conductive oxide layer to permit UV radiation to pass through conductive oxide layer;

FIG. 2B is a cross-sectional side view of the example electronic device of FIG. 2A, depicting additional features;

FIG. 3 is a schematic diagram of an example electronic device, depicting a UV light emitter and a UV light receiver to determine a touch-related biological contamination level of a transparent input interface;

FIG. 4A is a block diagram of an example electronic device including a non-transitory computer-readable storage medium, storing instructions to initiate a sterilization process to irradiate a surface of the electronic device; and

FIG. 4B is a block diagram of the example electronic device of FIG. 4A including a non-transitory computer-readable storage medium, storing instructions to control a time duration to perform the sterilization process.

### DETAILED DESCRIPTION

Electronic devices such as laptop computers, convertible devices, foldable smart phones, or the like may be used in different environments (e.g., an enterprise, a hospital, a home, an educational establishment, or the like). In such example environments, the electronic devices may be used by a user or shared by multiple users. Further, the electronic devices may include a user interface (e.g., a keyboard, a touchscreen display panel, a touch pad, and/or the like) to permit users to interact with the electronic devices. The user interface may be hand operated, where the users touch keys, buttons, and/or touchscreens with hand(s) and/or finger(s).

Consequently, contaminants present on the user's hand, including dirt, debris, bacteria, germs, fungus, viruses, and/or other pathogenic airborne microorganisms may transfer onto a surface of the electronic devices, for instance. In such a scenario, contamination and cross-contamination through the surface of the electronic devices may be a concern. The surface may be a mode of transmission of spreadable contaminants from one user to another user as the contaminants can stay active for significantly longer time on such surfaces (e.g., which are made up of a glass, plastic, and/or the like).

Some example electronic devices may be coated with nano-silver to protect the electronic devices from contamination. The nano-silver may possess a broad spectrum of antibacterial, antifungal, and antiviral properties. For example, the nano-silver may have the ability to penetrate bacterial cell walls, change the structure of cell membranes, and result in the cell death.

However, the use of nano-silver compounds may be hazardous for environment and/or for human health. By using such electronic devices coated with the nano-silver, the users may be breathing in the nano-silver. The nano-silver may have a toxic effect on human cells, suppressing cellular growth and multiplication, and causing the cell death. The nano-silver may also interfere with male sperm cell signals, causing sperm to stop growing, which in turn can affect male fertility. Further, the nano-silver can be toxic to lung cells, nerve cells and skin cells, and penetrates the brain flowing through blood circulation to other body tissues. Furthermore, while cleaning such electronic devices with water, the nano-silver may also flow into the natural environment with the water, interfering with the natural ecology. Furthermore, durability and service life of the electronic devices may be adversely affected because of nano-silver coatings. Also, silver ion concentration may be insufficient in such nano-silver coatings and thus, antibacterial effect may not be obvious.

Examples described herein may provide an electronic device including a display housing and a base housing pivotally connected to the display housing. Further, the base housing may include an input device having a transparent input interface exposed in an upper surface. Further, the base housing may include a transparent photocatalyst layer disposed on the transparent input interface. Furthermore, the base housing may include an ultraviolet (UV) light source disposed in the input device. Also, the base housing may include a conductive oxide layer disposed between the transparent input interface and the UV light source. Further, the electronic device may include a controller. When the display housing is in a closed position relative to the base housing, the controller may initiate a sterilization process by:
activating the UV light source to emit UV radiation, and
controlling a degree of transparency of the conductive oxide layer to direct the UV radiation at the transparent photocatalyst layer.

Thus, examples described herein utilize the UV light source with a photocatalyst (e.g., titanium dioxide (TIO₂)) to sterile the transparent input interface while the electronic device is in a clamshell mode (e.g., a configuration in which a display screen is facing a keyboard and the two are in parallel), thereby protecting the user from pathogenic microorganisms.

In the following description, for purposes of explanation, numerous specific details are set forth in order to provide a thorough understanding of the present techniques. However, the example apparatuses, devices, and systems, may be practiced without these specific details. Reference in the specification to “an example” or similar language means that a particular feature, structure, or characteristic described may be included in at least that one example but may not be in other examples.

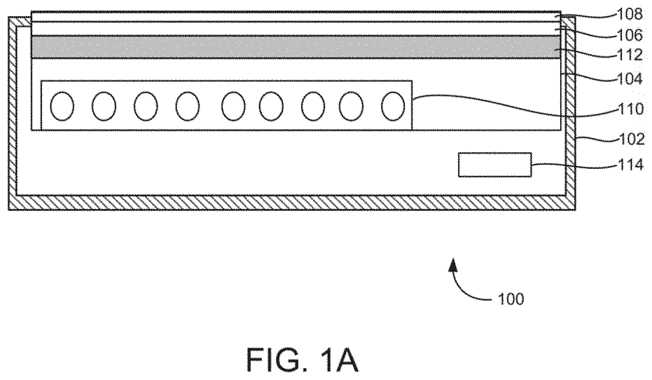

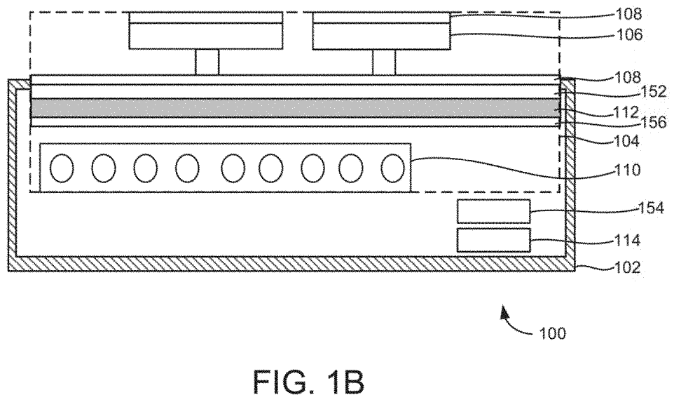

Turning now to the figures, FIG. 1A is a cross-sectional side view of an example electronic device 100, including a controller 114 to control a degree of transparency of a conductive oxide layer 112 to permit light to contact a transparent photocatalyst layer 108. Example electronic device 100 may include a notebook computer, a foldable smartphone, a convertible device, or the like. An example convertible device may refer to a device that can be “converted” from a laptop mode to a tablet mode.

As shown in FIG. 1A, electronic device 100 includes a housing 102. Further, electronic device 100 includes input device 104 exposed in an upper surface of housing 102. Furthermore, input device 104 includes a transparent input interface 106. In an example, input device 104 includes a mechanical keyboard having a transparent key cap, a touchscreen keyboard, a touchpad, or any combination thereof.

Further, electronic device 100 includes transparent photocatalyst layer 108 disposed on transparent input interface 106. In an example, transparent input interface 106 includes transparent photocatalyst layer 108 having antimicrobial properties for enhancing suppression of a biological activity. Transparent photocatalyst layer 108 may be used as a self-sterilization surface due to a property to form reactive oxygen species (ROS) when irradiated with ultraviolet (UV) light. The ROS may sterilize or disinfect transparent input interface 106 by attacking and inactivating microorganisms.

In an example, transparent photocatalyst layer 108 includes titanium dioxide (TiO₂), zinc oxide (ZnO), zirconium oxide (ZrO₂), tin oxide (SnO₂), tungsten trioxide (WO₃), strontium titanate (SrTiO₃), silicon dioxide (SiO₂), magnesium oxide (MgO), aluminum oxide (Al₂O₃), silicon nitride (Si₃N₄), magnesium fluoride (MgF₂), calcium fluoride (CaF₂), zinc Selenide (ZnSe) or zinc sulfide (ZnS), or any combination thereof.

Furthermore, electronic device 100 includes an UV light source 110 disposed in input device 104 to emit light. UV light source 110 may be disposed underneath transparent input interface 106. In an example, UV light source 110 includes a plurality of light emitting diodes (LEDs) to project UV-A light, UV-B light, UV-C light, or a combination thereof to transparent photocatalyst layer 108. In an example, UV light source 110 may be implemented as a part of a LED backlit keyboard. An example LED backlit keyboard is a red, green, blue (RGB) backlit keyboard that generates custom colors by projecting simultaneous combinations of red, green, and blue light, creating millions of potential hues.

Further, electronic device 100 includes conductive oxide layer 112 disposed in input device 104 between transparent input interface 106 and UV light source 110. In an example, conductive oxide layer 112 includes indium tin oxide (ITO), indium zinc oxide (IZO), or a combination thereof.

Furthermore, electronic device 100 includes controller 114. During operation, controller 114 may control a degree of transparency of conductive oxide layer 112 to permit the light to pass through conductive oxide layer 112 to contact transparent photocatalyst layer 108. In an example, controller 114 controls the degree of transparency of conductive oxide layer 112 by applying an input signal. Depending on the input signal, preferably an input voltage, the degree of transparency of conductive oxide layer 112 can be changed. ITO or IZO may provide a possibility to realize a transparent coating for conductive oxide layer 112 to be transparent.

In an example, controller 114 monitors a usage pattern of electronic device 100 over a period. The usage pattern may indicate a time interval that electronic device 100 is in a power-saving mode (e.g., a clamshell mode). For example, the usage pattern may include user login events to electronic device 100 (e.g., when the user logs into electronic device 100). In an example, the usage pattern of electronic device 100 may be monitored by analyzing data related to a login pattern selected from a group consisting of historical usage data, administrator-specified rules, or the like. Further, controller 114 may schedule to activate UV light source 110 to pass the light to transparent photocatalyst layer 108 based on the monitored usage pattern.

The combined action of UV light and photocatalytic materials such as TiO₂ may enhance the suppression of the biological activity on transparent input interface 106. The virucidal, bactericidal, fungicidal, or yeasticidal effect relies on the synergy between an inherent ability of the UV light to directly inactivate bacteria, fungus, virus, and/or other pathogenic airborne microorganisms through nucleic acid and protein damages, and the production of oxidative radicals generated through the irradiation of transparent photocatalyst layer 108.

FIG. 1B is a cross-sectional side view of example electronic device 100 of FIG. 1A, depicting additional features. For example, similarly named elements of FIG. 1B may be similar in structure and/or function to elements described with respect to FIG. 1A. In the example, shown in FIG. 1B, input device 104 may include a transparent top surface 152 including transparent input interface 106. For example, transparent input interface 106 is a surface of the backlit keyboard including a plurality of physical keys such as punctuation keys, alphanumeric and special keys like the Windows key, and different multimedia keys for entering input data into electronic device 100. In this example, transparent top surface 152 including transparent input interface 106 may be coated with transparent photocatalyst layer 108.

Further, electronic device 100 includes a transparent layer 156 disposed between conductive oxide layer 112 and UV light source 110. Furthermore, electronic device 100 includes a detector 154 to monitor a set of attributes relating to transparent input interface 106. For example, the set of attributes can include a frequency of the usage of electronic device 100, a presence of the biological activity on transparent input interface 106, a usage of electronic device 100, a disinfection schedule history for electronic device 100, and any combination thereof.

Further, based on the monitored set of attributes, controller 114 may control a degree of transparency of conductive oxide layer 112 to pass the light through conductive oxide layer 112 to contact transparent photocatalyst layer 108 on transparent top surface 152 and transparent input interface 106. Further, controller 114 may operate UV light source 110 to emit the light for a period, for instance, based on the monitored set of attributes. In other examples, controller 114 can control an activation of UV light source 110 to sterilize a portion of transparent input interface 106 based on an area of contamination.

In an example, the components of electronic device 100 may be implemented in hardware, computer-readable instructions, or a combination thereof. In an example, controller 114 and detector 154 may be implemented as engines or modules comprising any combination of hardware and programming to implement the functionalities described herein. The functions of controller 114 and detector 154 can be implemented using a processor.

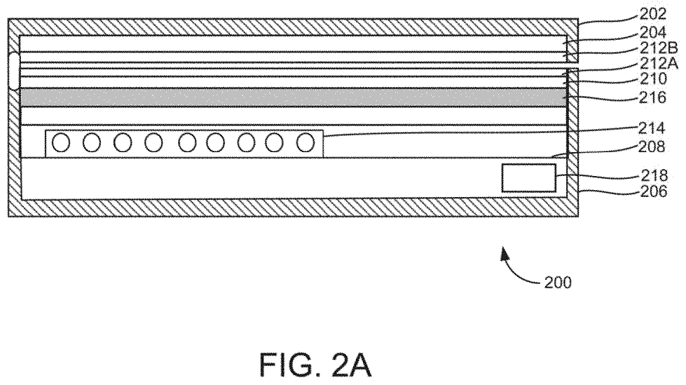

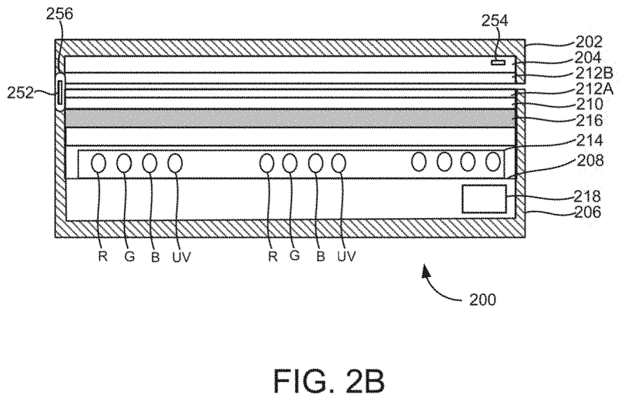

FIG. 2A is a cross-sectional side view of an example electronic device 200, including a controller 218 to control a degree of transparency of a conductive oxide layer 216 to permit UV radiation to pass through conductive oxide layer 216. As shown in FIG. 2A, electronic device 200 includes a display housing 202 to house a display panel 204 (e.g., a touchscreen display panel). Example display panel 204 includes a liquid crystal display (LCD), light emitting diode (LED), electro-luminescent (EL) display, or the like. Further, electronic device 200 includes a base housing 206 to pivot between an open position and a closed position relative to display housing 202. For example, base housing 206 may house a keyboard, a battery, a touchpad, and so on. In other examples, display housing 202 and base housing 206 may house other components such as a camera, audio/video devices, and the like, depending on the functions of electronic device 200.

Further, electronic device 200 includes an input device 208 (e.g., a keyboard, a touchpad, or the like) exposed in an upper surface of base housing 206. Further, input device 208 includes a transparent input interface 210. Furthermore, electronic device 200 includes a first transparent photocatalyst layer 212A disposed on transparent input interface 210 and a second transparent photocatalyst layer 212B disposed on display panel 204. In an example, first transparent photocatalyst layer 212A and second transparent photocatalyst layer 2128 include TiO₂, ZnO, ZrO₂, SnO₂, WO₃, SrTiO₃, SiO₂, MgO, Al₂O₃, Si₃N₄, MgF₂, CaF₂, ZnSe, ZnS, or any combination thereof.

For example, TiO₂ is an antimicrobial material and is used as first transparent photocatalyst layer 212A and second transparent photocatalyst layer 2128 for different purposes including removal of organic and inorganic compounds and inactivation of harmful microorganisms on a surface of transparent input interface 210 and display panel 204. TiO₂, when energized by UV light (e.g., backlight) of input device 208, forms reactive oxygen species that can attack organic compounds, break apart the organic compounds' chemical bonds, and turn the organic compounds into harmless substances such as carbon dioxide and water.

Further, electronic device 200 includes a UV light source 214 disposed in input device 208. Furthermore, electronic device 200 includes conductive oxide layer 216 disposed between transparent input interface 210 and UV light source 214. In an example, conductive oxide layer 216 includes indium tin oxide (ITO), indium zinc oxide (IZO), or a combination thereof.

Further, electronic device 200 includes controller 218. During operation, controller 218 may control, when base housing 206 is in the closed position, a degree of transparency of conductive oxide layer 216 to direct UV radiation from UV light source 214 at first transparent photocatalyst layer 212A and second transparent photocatalyst layer 212B.

In an example, controller 218 may detect a trigger event related to closing of base housing 206 relative to display housing 202. Further, controller 218 may calculate a set of time intervals that electronic device 200 is in the closed position over a period. Furthermore, controller 218 may enable a user to select a time interval from the set of time intervals to activate UV light source 214 to sterilize transparent input interface 210 and display panel 204. Further, controller 218 may sterilize transparent input interface 210 and display panel 204 according to the selected time interval.

FIG. 2B is a cross-sectional side view of example electronic device 200 of FIG. 2A, depicting additional features. For example, similarly named elements of FIG. 2B may be similar in structure and/or function to elements described with respect to FIG. 2A. As shown in FIG. 2B, electronic device 200 includes a hinge 256 to pivotally connect base housing 206 and display housing 202.

Further, electronic device 200 includes a first sensor 252 to determine the closed position of base housing 206 relative to display housing 202. In an example shown in FIG. 2B, first sensor 252 may include a lid angle sensor disposed in hinge 256 between display housing 202 and base housing 206 to determine an opening and closing of display housing 202. In yet another example, first sensor 252 may be a magnetic sensor (e.g., a Hall-effect sensor) in a palm rest area of base housing 206 and is used with a magnet in display housing 202. When the magnetic sensor and the magnet get close, the magnetic sensor trips to indicate a clamshell mode. In another example, first sensor 252 may be a switch located in or around hinge 256, which is pressed when display housing 202 is closed.

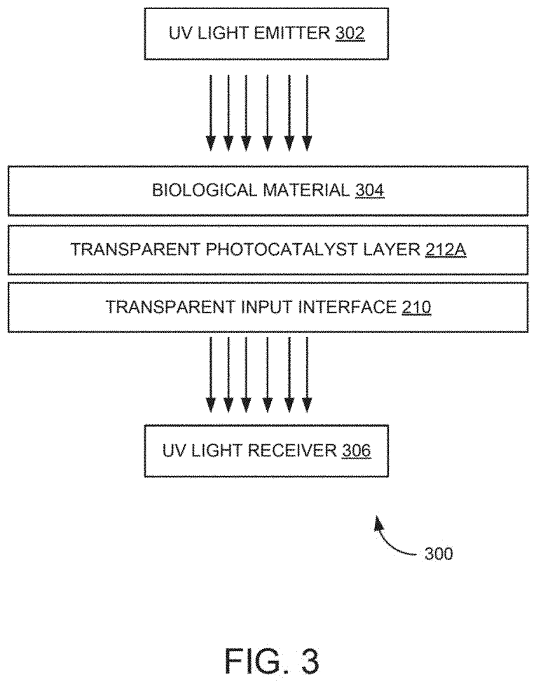

Further, electronic device 200 includes a second sensor 254 to determine a touch-related contamination level of display panel 204, transparent input interface 210, or a combination thereof when base housing 206 is in the closed position. For example, second sensor 254 is a web camera disposed in display housing 202. An example second sensor 254 is described with respect to FIG. 3.

FIG. 3 is a schematic diagram of an electronic device 300, depicting a UV light emitter 302 and a UV light receiver 306 to determine a touch-related biological contamination level of transparent input interface 210. In the example shown in FIG. 3, a second sensor (e.g., second sensor 254 of FIG. 2B) includes UV light emitter 302 and UV light receiver 306. UV light emitter 302 and UV light receiver 306 may be disposed in a display housing (e.g., display housing 202 of FIG. 2B) and a base housing (e.g., base housing 206 of FIG. 2B), respectively, or vice versa.

During operation, UV light emitter 302 may emit visible light to be incident on transparent photocatalyst layer 212A of transparent input interface 210 in response to detecting that display housing 202 is moved to a closed position. Further, UV light receiver 306 may detect the visible light emitted from UV light emitter 302 and progressing through transparent input interface 210. Furthermore, a controller (e.g., controller 218 as shown in FIG. 2B) may estimate a protein concentration of a biological material 304 on transparent input interface 210 based on an amount of the visible light absorbed by transparent input interface 210. Similarly, the protein concentration of biological material 304 on both transparent input interface 210 and a display panel (e.g., display panel 204 of FIG. 2B) can be estimated.

For example, a significant material of bacteria and virus is the protein. The protein absorbs the visible light (e.g., UVC light) at specific wavelengths (e.g., protein absorption peaks at about 280 nm and nucleic acids at about 260 nm). The amount of protein absorbed may be used to estimate the protein and dirt concentration on transparent input interface 210 and is used as an index to turn on/off the UV sterilization.

For example, when UV light emitter 302 irradiates an organic compound (i.e., biological material 304), transparent input interface 210 may absorb the UVC light if transparent input interface 210 contains the organic compound of unsaturated bonding such as hydrocarbons and benzene rings. Using UV light receiver 306, the amount of UVC light absorbed by transparent input interface 210 can be detected.

A significant increase in the organic ingredients in unsaturated bonds on transparent input interface 210 may significantly increase the absorption of the UVC light. If organic matter contains only unsaturated organic matter with a single component, then UVC absorption energy is proportional to organic composition. A change in energy (E) can be calculated by converting the light decay value to an electronic signal as shown below.

E=hv,
where, h is the Planck constant and v is the frequency of light.

The value of the Planck constant is approximately: h=6.6260693(11)×10⁻³⁴ J s. The Energy loss ratio can be obtained using the following equation:

Energy loss ratio=[UVC(in)−UVC(out)]/UVC(in),
where, UVC (in) refers to an amount of UVC light emitted by UV light emitter 302 and UVC (out) refers to an amount of light received by UV light receiver 306.

The change in energy is used to determine the amount of the UVC light absorbed by transparent input interface 210. Referring back to FIG. 2B, in response to determining the closed position and the touch-related contamination level, controller 218 may activate UV light source 214 to emit the UV radiation. In the example shown in FIG. 2B, UV light source 214 may include red, green, blue (RGB) LEDs and UV LEDs. Further, controller 218 may control a degree of transparency of conductive oxide layer 216 to direct the UV radiation at first transparent photocatalyst layer 212A and second transparent photocatalyst layer 212B.

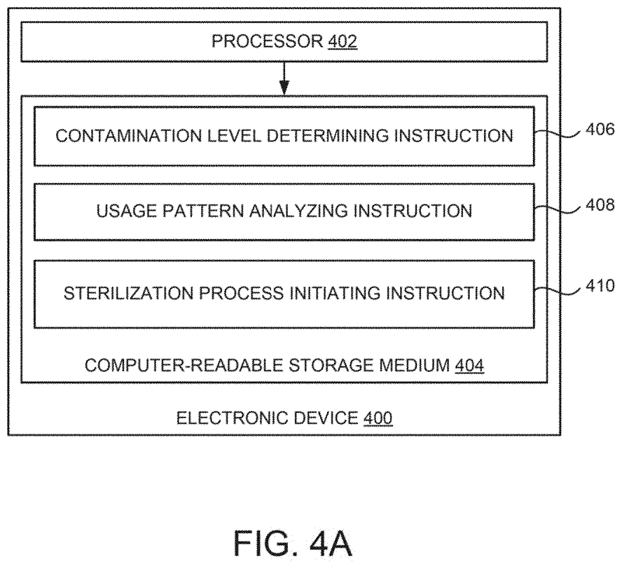

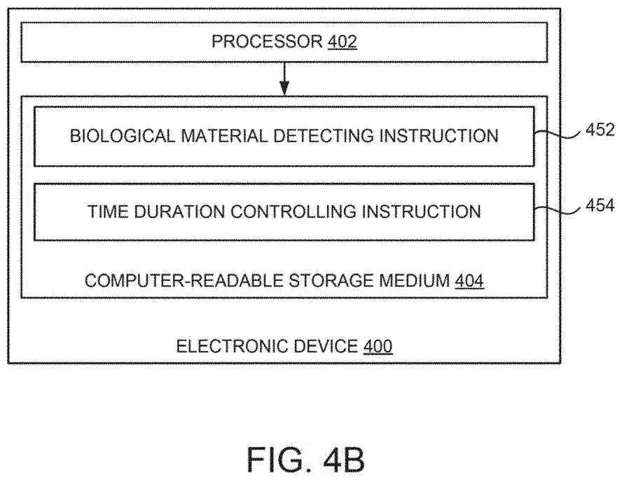

FIG. 4A is a block diagram of an example electronic device 400 including a non-transitory computer-readable storage medium 404, storing instructions to initiate a sterilization process to irradiate a surface of electronic device 400. Example electronic device 400 may be a laptop computer, a foldable smartphone, or any other device having a touch-based input surface. In an example, electronic device 400 includes a display housing pivotally connected to a base housing.

Electronic device 400 may include a processor 402 and computer-readable storage medium 404 communicatively coupled through a system bus. Processor 402 may be any type of central processing unit (CPU), microprocessor, or processing logic that interprets and executes computer-readable instructions stored in computer-readable storage medium 404.

Computer-readable storage medium 404 may be a random-access memory (RAM) or another type of dynamic storage device that may store information and computer-readable instructions that may be executed by processor 402. For example, computer-readable storage medium 404 may be synchronous DRAM (SDRAM), double data rate (DDR), Rambus® DRAM (RDRAM), Rambus® RAM, and the like, or storage memory media such as a floppy disk, a hard disk, a CD-ROM, a DVD, a pen drive, and the like. In an example, computer-readable storage medium 404 may be a non-transitory computer-readable medium, where the term “non-transitory” does not encompass transitory propagating signals. In an example, computer-readable storage medium 404 may be remote but accessible to electronic device 400.

Computer-readable storage medium 404 may store instructions 406, 408, and 410. Instructions 406 may be executed by processor 402 to determine a touch-related biological contamination level of a surface of electronic device 400. In an example, instructions 406 to determine the touch-related biological contamination level of the surface of electronic device 400 include instructions to determine the touch-related biological contamination level of a surface of a display panel disposed in the display housing, a top surface of the base housing including a keyboard, or a combination thereof.

Further, instructions 406 to determine the touch-related biological contamination level of the surface may include instructions to estimate a protein concentration of a biological material on the surface of electronic device 400 via a UV light sensor disposed in electronic device 400. For example, instructions to estimate the protein concentration of the biological material include instructions to:
emit visible light, via a light emitter of the UV light sensor, to be incident on the surface in response to detecting that the display housing is moved to the closed position,
detect the visible light, via a light receiver of the UV light sensor, emitted from the light emitter and progressing through the surface, and
estimate the protein concentration of the biological material based on an amount of the visible light absorbed by the surface.

The light emitter and the light receiver may be disposed in the display housing and the base housing, respectively, or vice versa. Instructions 408 may be executed by processor 402 to analyze a usage pattern of electronic device 400 to determine a time interval to perform the sterilization process upon determining the touch-related biological contamination level. In some examples, computer-readable storage medium 404 may store instructions to generate an alert notification seeking a user selection of the time interval to initiate the sterilization process. In this example, processor 402 may analyze a usage pattern of electronic device 400 to determine a set of time intervals to perform the sterilization process and the set of time intervals may be presented to the user via the alert notification.

Instructions 410 may be executed by processor 402 to initiate the sterilization process in response to detecting that the display housing is moved to a closed position relative to the base housing. The sterilization process may be performed to irradiate the surface using a UV source based on the usage pattern and the touch-related biological contamination level. In an example, instructions 410 to sterilize the surface include instructions to sterilize the surface to inactivate the biological material selected from a group consisting of bacteria, viruses, yeasts, and fungi. Further, instructions 410 to initiate the sterilization process include instructions to:
adjust voltage to control a degree of transparency of a conductive oxide layer disposed between the surface and UV light source, and
direct a UV radiation from the UV light source to a transparent photocatalyst layer disposed on the surface via the conductive oxide layer.

In some examples, the surface includes the transparent photocatalyst layer having antibacterial properties, such as TiO₂, which can assist in sterilization and disinfection and is activated by light. During the sterilization process, the degree of transparency of the conductive oxide layer may be controlled to greater than or equal to a first threshold (e.g., 80%), in which the UV radiation can pass through the conductive oxide layer. Upon completion of the sterilization process, the degree of transparency of the conductive oxide layer may be controlled to less than or equal to a second threshold (e.g., 30%).

FIG. 4B is a block diagram of example electronic device 400 of FIG. 4A including a non-transitory computer-readable storage medium 404, storing instructions to control a time duration to perform the sterilization process. For example, similarly named elements of FIG. 4B may be similar in structure and/or function to elements described with respect to FIG. 4A.

Computer-readable storage medium 404 may store instructions 452 and 454. In an example, instructions 452 may be executed by processor 402 to detect a type of biological material based on the estimated protein concentration. Further, instructions 454 may be executed by processor 402 to control a time duration to perform the sterilization process based on the type of biological material and the determined time interval. In some examples, a Basic Input Output System (BIOS) of electronic device 400 may control the time duration to do the sterilization process.

In an example, consider that the energy value of the surface when the surface is clean as C0. When the display housing is closed, the conductive oxide layer may be turned to transparent and the UV light source may be triggered to get the energy value of the surface, for instance, C1. When C0-C1 is greater than a predefined minimum value, consider a dirty index (e.g., a measure of biological contamination) as 25. When C0-C1 is greater than a predefined maximum value, consider the dirty index as 50. Also, consider that the user selects a time index (e.g., a measure of clamshell mode duration) to do the sterilizing process as follows:

time index=100 for first priority,

time index=75 for second priority,

time index=50 for third priority, and

time index=25 for fourth priority.

Based on the dirty index and the time index, electronic device 400 may initiate an example sterilization process as follows:
When the dirty index is 50 and the time index is 100, initiate the sterilization process.
When the dirty index is 50 and the time index is 50, pop-up a message notifying the user to close the display housing to proceed with the sterilization process.
When dirty index is 25 and best time is 50, then a counter is initiated to count a number of times the dirty index is measured as 25 and the best time is measured as 50. When a value of the counter is greater than 3, then the sterilization process is triggered.
When the dirty index is 25 and the time index is 25, then the sterilization process is not triggered.

The above-described examples are for the purpose of illustration. Although the above examples have been described in conjunction with example implementations thereof, numerous modifications may be possible without materially departing from the teachings of the subject matter described herein. Other substitutions, modifications, and changes may be made without departing from the spirit of the subject matter. Also, the features disclosed in this specification (including any accompanying claims, abstract, and drawings), and/or any method or process so disclosed, may be combined in any combination, except combinations where some of such features are mutually exclusive.

The terms “include,” “have,” and variations thereof, as used herein, have the same meaning as the term “comprise” or appropriate variation thereof. Furthermore, the term “based on”, as used herein, means “based at least in part on.” Thus, a feature that is described as based on some stimulus can be based on the stimulus or a combination of stimuli including the stimulus. In addition, the terms “first” and “second” are used to identify individual elements and may not meant to designate an order or number of those elements.

The present description has been shown and described with reference to the foregoing examples. It is understood, however, that other forms, details, and examples can be made without departing from the spirit and scope of the present subject matter that is defined in the following claims.

What is claimed is:

1. An electronic device comprising:
a housing;
an input device exposed in an upper surface of the housing, the input device comprising a transparent input interface;
a transparent photocatalyst layer disposed on the transparent input interface;
an ultraviolet (UV) light source disposed in the input device to emit light;
a conductive oxide layer disposed in the input device between the transparent input interface and the UV light source; and
a controller to control a degree of transparency of the conductive oxide layer to permit the light to pass through the conductive oxide layer to contact the transparent photocatalyst layer.

2. The electronic device of claim 1, wherein the transparent photocatalyst layer comprises titanium dioxide (TiO₂), zinc oxide (ZnO), zirconium oxide (ZrO₂), tin oxide (SnO₂), tungsten trioxide (WO₃), strontium titanate (SrTiO₃), silicon dioxide (SiO₂), magnesium oxide (MgO), aluminum oxide (Al₂O₃), tin oxide (SnO₂), silicon nitride (Si₃N₄), magnesium fluoride (MgF₂), calcium fluoride (CaF₂), zinc selenide (ZnSe), zinc sulfide (ZnS), or any combination thereof.

3. The electronic device of claim 1, wherein the UV light source comprises a plurality of light emitting diodes (LEDs) to project UV-A light, UV-B light, UV-C light, or a combination thereof to the transparent photocatalyst layer.

4. The electronic device of claim 1, wherein the conductive oxide layer comprises indium tin oxide (ITO), indium zinc oxide (IZO), or a combination thereof.

5. The electronic device of claim 1, wherein the input device comprises a mechanical keyboard having a transparent key cap, a touchscreen keyboard, a touchpad, or any combination thereof.

6. The electronic device of claim 1, further comprising:
a detector to monitor a set of attributes relating to the transparent input interface, wherein, based on the monitored set of attributes, the controller is to:
control a degree of transparency of the conductive oxide layer to pass the light through the conductive oxide layer to contact the transparent photocatalyst layer; and
operate the UV light source to emit the light for a period.

7. The electronic device of claim 1, wherein the controller is to:
monitor a usage pattern of the electronic device over a period, the usage pattern indicating a time interval that the electronic device is in a power-saving mode; and
schedule to activate the UV light source to pass the light to the transparent photocatalyst layer based on the monitored usage pattern.

8. An electronic device comprising:
a display housing to house a display panel;
a base housing to pivot between an open position and a closed position relative to the display housing;
an input device exposed in an upper surface of the base housing, the input device comprising a transparent input interface;
a first transparent photocatalyst layer disposed on the transparent input interface;
a second transparent photocatalyst layer disposed on the display panel;
an ultraviolet (UV) light source disposed in the input device;
a conductive oxide layer disposed between the transparent input interface and the UV light source; and
a controller to control, when the base housing is in the closed position, a degree of transparency of the conductive oxide layer to direct UV radiation from the UV light source at the first transparent photocatalyst layer and the second transparent photocatalyst layer.

9. The electronic device of claim 8, wherein the first transparent photocatalyst layer and the second transparent photocatalyst layer comprise titanium dioxide (TiO₂), zinc oxide (ZnO), zirconium oxide (ZrO₂), tin oxide (SnO₂), tungsten trioxide (WO₃), strontium titanate (SrTiO₃), silicon dioxide (SiO₂), magnesium oxide (MgO), aluminum oxide (Al₂O₃), tin oxide (SnO₂), silicon nitride (Si₃N₄), magnesium fluoride (MgF₂), calcium fluoride (CaF₂), zinc selenide (ZnSe), zinc sulfide (ZnS), or any combination thereof.

10. The electronic device of claim 8, wherein the conductive oxide layer comprises indium tin oxide (ITO), indium zinc oxide (IZO), or a combination thereof.

11. The electronic device of claim 8, further comprising:
a first sensor to determine the closed position of the base housing relative to the display housing; and
a second sensor to determine a touch-related contamination level of the display panel, the transparent input interface, or a combination thereof when the base housing is in the closed position, wherein, in response to determining the closed position and the touch-related contamination level, the controller is to:
activate the UV light source to emit the UV radiation; and
control a degree of transparency of the conductive oxide layer to direct the UV radiation at the first transparent photocatalyst layer and the second transparent photocatalyst layer.

12. The electronic device of claim 8, wherein the controller is to:
detect a trigger event related to closing of the base housing relative to the display housing;
calculate a set of time intervals that the electronic device is in the closed position over a period; and
enable to select a time interval from the set of time intervals to activate the UV light source to sterilize the transparent input interface and the display panel.

13. A non-transitory computer-readable storage medium storing instructions executable by a processor of an electronic device to:
determine a touch-related biological contamination level of a surface of the electronic device, the electronic device comprising a display housing pivotally connected to a base housing;
upon determining the touch-related biological contamination level, analyze a usage pattern of the electronic device to determine a time interval to perform a sterilization process; and
in response to detecting that the display housing is moved to a closed position relative to the base housing, initiate the sterilization process to irradiate the surface using an ultraviolet light (UV) source based on the usage pattern and the touch-related biological contamination level.

14. The non-transitory computer-readable storage medium of claim 13, wherein instructions to initiate the sterilization process comprise instructions to:
adjust voltage to control a degree of transparency of a conductive oxide layer disposed between the surface and UV light source; and
direct a UV radiation from the UV light source to a transparent photocatalyst layer disposed on the surface via the conductive oxide layer.

15. The non-transitory computer-readable storage medium of claim 13, wherein instructions to determine the touch-related biological contamination level of the surface comprise instructions to:
estimate a protein concentration of a biological material on the surface of the electronic device via a UV light sensor disposed in the electronic device.

16. The non-transitory computer-readable storage medium of claim 15, wherein instructions to estimate the protein concentration of the biological material comprise instructions to:
emit visible light, via a light emitter of the UV light sensor, to be incident on the surface in response to detecting that the display housing is moved to the closed position;
detect the visible light, via a light receiver of the UV light sensor, emitted from the light emitter and progressing through the surface, wherein the light emitter and the light receiver are disposed in the display housing and the base housing, respectively, or vice versa; and
estimate the protein concentration of the biological material based on an amount of the visible light absorbed by the surface.

17. The non-transitory computer-readable storage medium of claim 15, further comprising instructions to:
detect a type of biological material based on the estimated protein concentration; and
control a time duration to perform the sterilization process based on the type of biological material and the determined time interval.

18. The non-transitory computer-readable storage medium of claim 13, wherein instructions to determine the touch-related biological contamination level of the surface of the electronic device comprise instructions to:
determine the touch-related biological contamination level of a surface of a display panel disposed in the display housing, a top surface of the base housing including a keyboard, or a combination thereof.

19. The non-transitory computer-readable storage medium of claim 13, wherein instructions to sterilize the surface comprise instructions to sterilize the surface to inactivate the biological material selected from a group consisting of bacteria, viruses, yeasts, and fungi.

20. The non-transitory computer-readable storage medium of claim 13, further comprising instructions to:
generate an alert notification seeking a user selection of the time interval to initiate the sterilization process.
